(19) United States
(12) Patent Application Publication
(10) Pub. No.: US 2017/0014604 A1
(43) Pub. Date: Jan. 19, 2017

(54) Iris Dilator

(71) Applicant: Francis Y. Falck, JR., Stonington, CT (US)

(72) Inventor: Francis Y. Falck, JR., Stonington, CT (US)

(21) Appl. No.: 14/800,744

(22) Filed: Jul. 16, 2015

(51) Int. Cl. A61M 29/00; A61F 9/007; A61B 17/02

(52) U.S. Cl. CPC A61M 29/00; A61B 17/0231; A61F 9/007

(57) ABSTRACT

An iris dilator has a head spring transmitting a spring bias to a pair of limbs extending from the head spring. The limbs are provided with loops that can engage an inner perimeter of an iris while the bias exerted by the head spring results in a dilation occurring approximately at the four corners of an iris opening.

### TECHNICAL FIELD

Surgical iris dilators.

### BACKGROUND

For cataract surgery, and other surgeries inside the eye, the surgeon must have an unobstructed view. To accomplish this the iris pupil must be dilated. Some pupils will not dilate due to the side effects of systemic medications, trauma and/or scar tissue. These pupils need to be mechanically stretched open.

There have been suggestions in the prior art for iris pupil dilators, but these are cumbersome, require multiple incisions, need special insertion and removal tools, overstretch the pupil causing irregularity, have uncontrolled expansion and increased surgery time which increases the risk of surgery. A sample of such prior art includes: U.S. Pat. Nos. 4,257,406; 5,607,446; 6,183,480; 8,323,296; 8,439,833; and 8,496,583.

### SUMMARY

This application proposes solutions that are mechanically simple and self-actuated, to automatically open a pupil to a desired size. One proposal dilates a pupil to the approximate size of a square about 6 mm on each side. The square proposal can be done with a pair of opposed devices or with a single device. The opposed devices are inserted through a pair of opposite incisions, and the single device is inserted through a single incision so that either proposal can dilate the iris to a four-sided square opening. These proposals preferably eliminate any hooks engaging an inner perimeter of an iris. Although hooks can work for this, they can also catch onto tissue in ways not intended so that they become more difficult and time consuming to manage.

Preferred dilator devices have spring limbs in sufficient numbers to dilate an inner perimeter of an iris enough to give a surgeon a clear view of what is happening. Spring limbs devised to dilate an iris at four corner positions is sufficient enough so that increasing the spring limb engagements with the iris to 5, 6, or more positions adds undesirable complexity.

The solutions that are preferred for safety and expediency are made of a flexible plastic material such as polypropylene. This material can be engineered to apply spring biases of calculated amounts applied in different regions of the devices to ensure trouble-free operation. The preferred spring material is also formed as a head spring applying a predetermined bias to spread apart an opposed pair of spring limbs extending from the spring head. The spring bias is sufficient to spread the limbs apart with sufficient force to dilate an inner perimeter of an iris. Besides having a gentle spring bias that is suitable for spreading spring limbs, the solution also preferably includes a way of inter-engaging spring limbs to hold the device in a compact configuration allowing insertion through an incision in an eye.

### DRAWINGS

FIG. 1 is a schematic view of a 4-sided dilator powered by a head spring.

FIG. 2 is a schematic view of the dilator of FIG. 1 with spring limbs being drawn together and inter-engaged in a compact size that can fit through a 2-3 mm incision.

FIG. 3 is a schematic view of a pair of spring limbs biased by a head spring to urge the spring limbs apart. A pair of the dilators illustrated in FIG. 3 can be inserted via separate incisions on opposite sides of an eye to produce four engagement loops forcing an iris dilation of an approximately square size.

FIGS. 4 and 5 are side views of a dilator similar to the one illustrated in FIG. 2 to show locking and unlocking of the distal end of opposed limbs and to show how loops on opposed limbs are inclined from a plane of the limbs.

### DETAILED DESCRIPTION

The dilators that are schematically illustrated in the drawings are made of a flexible material such as spring wire or polymer. Spring wire has been used for prototype design and has advantages in quick approximations made for test purposes. The wire can be given a permanent bend and can be used in single or double reaches for varying the transmission of spring bias to the iris.

A molded plastic material such as polypropylene has several advantages that make it preferred over spring wire. These advantages include inexpensiveness sufficient for discarding the dilator after each use. A molded polymer can also vary the thickness and structural strength that the spring bias transmits to the iris. This assures that the dilation job is done expediently without harming the iris.

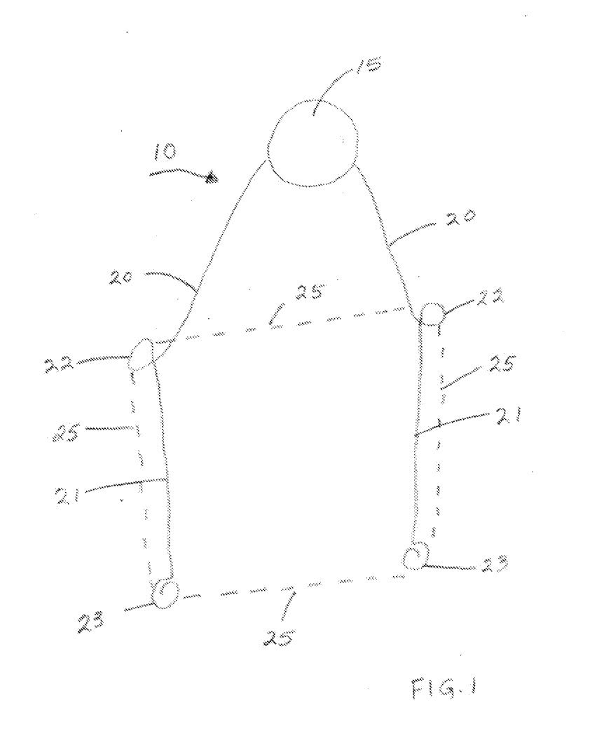

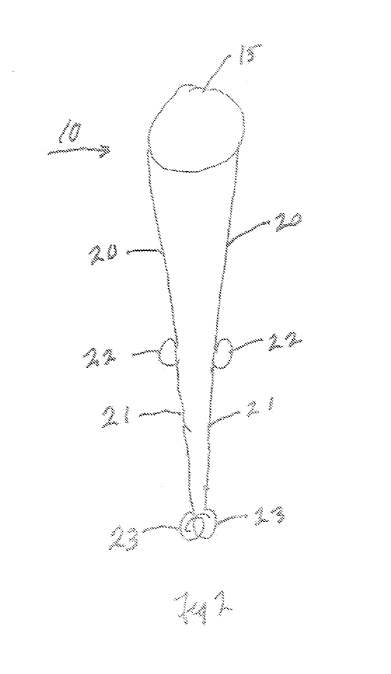

Dilator 10, as schematically illustrated in FIGS. 1 and 2, includes a head spring 15, and an opposed pair of spring limbs 20 and 21. Head spring 15 exerts a spring bias force against limbs 20 to spread them apart as shown in FIG. 1. Limbs 20 and 21 are provided with loops 22 and 23 to engage an inner perimeter of an iris at four locations arranged at corners of a square. The tissue of the inner perimeter of an iris is shown in broken lines 25.

The actual size of dilator 10 is tiny compared with the illustration in FIGS. 1 and 2. Dilator 10 preferably folds limbs 20 and 21 into a compact, juxtaposed position shown in FIG. 2, for insertion into an eye. Limb loops 23 are shaped to inner-engage each other in a hook-type of arrangement holding dilator 10 to the position shown in FIG. 2 for insertion into an eye. Limb loops 23, as shown in FIG. 2, have a hook-type inner-lock holding dilator 10 in the position illustrated in FIG. 2 for insertion into an incision in an eye. Dilator 10 can be released from the compact position illustrated in FIG. 2 simply by squeezing limbs 20 toward each other a little way below spring head 15. Better yet, with spring head 15 being gripped in forceps for insertion into an eye, the resistance occurring from thrusting the loops forward through the incision region is enough to unhook loops 23. This results in expansion of dilator 10 from the position shown in FIG. 2 to the working position shown in FIG. 1. Loops 22 and 23 bear against and engage an inner periphery 25 of an iris of an eye to dilate the iris to the spread open position of FIG. 1. This leaves spring head 15 outside of the eye for later removal of the device from the eye. The spring head nevertheless provides biasing force spreading limbs 20 and 21 apart, as shown in FIG. 1.

Although loops 23 on spring arms 21 are capable of inter-engaging in a latch holding dilator 10 to the position of FIG. 2, loops 23 do not hook onto the material of the iris or of the eye itself. This avoids any need for unhooking dilator 10 from any undesirable entanglement with eye tissue.

This also helps expedite a surgeon's work. After the suitable incision is made, head spring 15 can be gripped with forceps to thrust the limbs through the incision, which tends to unhook loops 23 and allow dilator 10 to move from the spring bias resistance position of FIG. 2 to the spring fully biased position of FIG. 1. If the opening and spreading apart of limbs 20 and 21 does not occur as planned, then a squeeze applied to limbs 20 near spring head 15 can accomplish the necessary unhooking to allow dilator 10 to expand from the position of FIG. 2 to the position of FIG. 1. The expansion of limbs and limb feet automatically applies the spring bias to spreading the loops 22 and 23 into a square dilation of the iris without involving any excess force.

Dilator 10 is preferably dimensioned and designed to cooperate with a single incision made in an eye. This is preferably made from 2 to 3 mm in length, which is sufficient to squeeze the limbs together as shown in FIG. 2. As the dilator advances through the incision, it is squeezed to hold the leading loops in their compact and interlocking position as the insertion advances. Before the dilator is completely expanded to the position of FIG. 1, it releases from the squeeze supplied by the incision so that the limbs can separate, and the loops 22 and 23 can spread apart against an inner perimeter of an eye. A pinch with forceps can make this spreading apart occur if the incision does not succeed in this. The limited width of the incision also squeezes the limbs toward each other as dilator 10 is withdrawn from the eye through the incision. Here again, a squeeze from forceps can help the process along.

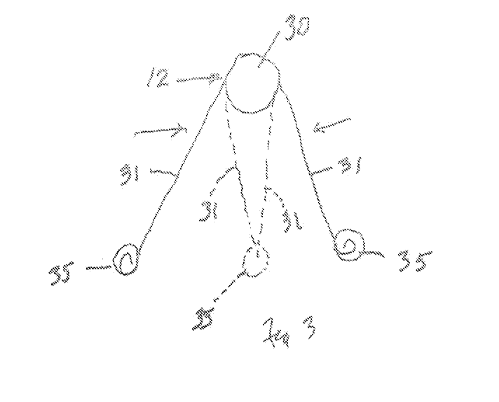

The embodiment 12, illustrated in FIG. 3 includes a head spring 30 and spring limbs 31 terminating in spring loops 35. These can be formed with terminal hooks or interlocks so that dilator 12 can be squeezed as suggested by the arrows, to the latched position shown in broken lines. This can be beneficial when it is desirable to make a pair of opposed incisions and insert a pair of dilators 12 through the incisions and against the inner perimeter of an iris by the terminal loops 35. Loops 35 initially spread a portion of the internal perimeter of an iris by the amount shown in FIG. 3. Another dilator 12 can then be inserted through an opposite incision until its terminal limb loops 35 are spaced apart by the same distance. The four loops 35 are then positioned approximately at the corners of a square to provide a surgeon with an adequate view. When the surgery is completed, dilators 12 can each be removed from the incisions into which they were inserted. The system presented by dilators 12 requires two incisions and two dilators 12, so that it is a little more complex and time consuming. It may be useful, nevertheless, for special circumstances.

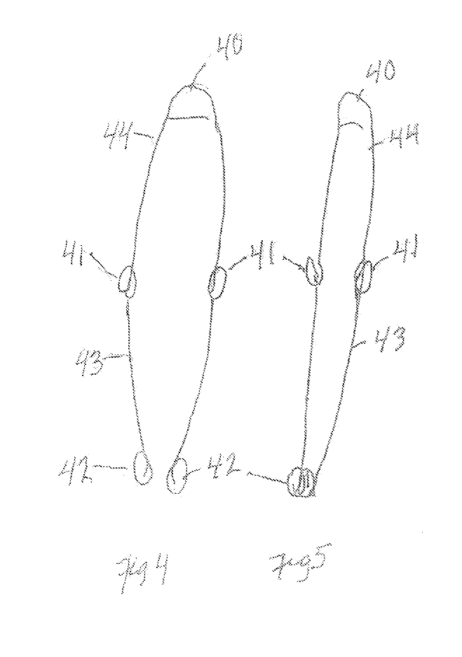

FIGS. 4 and 5 illustrate how closed limb loops 41 and open limb loops 42 each accomplish the desired functions. Loops 41 and 42 are preferably inclined relative to a plane of limbs 43. This gives the loops an aspect that is somewhat oblique to limb plane 43 so that loops can reliably engage an inner perimeter of an iris without slipping into a space above or below the iris. The inclined aspect of the loops relative to the limbs gives the loops an apparent thickness to ensure proper engagement and dilation of an inner perimeter of an iris.

The open loops 42 at the distal end regions of limbs 44 provide an interlocking engagement that holds loops 42 to each other without disposing a hook to face and possibly attach to iris tissue. The capacity for loops 42 to hook to each other as illustrated in FIGS. 4 and 5 can be unhooked by squeezing limbs 44 near head spring 40. This preferably occurs naturally while limbs 44 are being inserted into an eye, but it can also be accomplished with forceps.

When it occurs naturally, the width of the incisions through which the loops are inserted plays a role. It squeezes the limbs toward each other before they are fully inserted, so as to unlock the loops and allow the limbs to expand fully to the positions illustrated in FIGS. 4 and 5.

What is claimed is:

1. An iris dilator comprising:
a length of spring material having a central region formed as a head spring and a pair of opposed spring limbs extending from the head spring;
the head spring being structured to apply a spring bias to the limbs to urge the limbs apart;
the limbs have loops arranged to engage an inner perimeter of an iris;
opposing loops at digital ends of the limbs being opened to interlock with each other to hold distal ends of the loops together; and
the distal end loops being arranged to unlock from each other during insertion of the dilator into an eye.

2. The dilator of claim 1 wherein the dilator is molded of polymeric spring material shaped to exert different edge spring biases in different regions of the dilator.

3. The dilator of claim 1 wherein feet of the limbs are configured to hook to each other during insertion of the limbs into the eye.

4. The dilator of claim 1 wherein the loops are inclined relative to a plane of the limbs.

5. An iris dilator comprising:
a pair of spring material limbs extending from a head spring in a central region of a length of the spring material;
the head spring being deployed to bias the limbs apart;
the limbs having loops positioned to engage an inner perimeter of an iris while the limbs are biased apart by the head spring;
distal regions of the limbs having open loops arranged to interlock to hold the dilator in a compact position;
the interlock between the distal regions of the limbs being releasable to allow the limbs and their loops to spread apart within the eye; and
the bias of the head spring being applied to separate the limbs and their loops so that the loops engage an inner perimeter region of an iris with a spring bias that dilates the iris.

6. The dilator of claim 5 wherein the dilator is molded of polymeric spring material shaped to exert different edge spring biases in different regions of the dilator.

7. The dilator of claim 5 wherein the latching of the limbs is arranged to disengage during the insertion of the limbs into the eye.

8. The dilator of claim 5 wherein the loops are inclined relative to a plane of the limbs.

9. An iris dilator comprising:
a length of spring material having a central region formed as a head spring;
a pair of opposed limbs extending from the head spring;
the head spring being configured to apply a spring bias to the limbs to urge the limbs apart;
the limbs being held in juxtaposition by an inter-engagement that prevents the limbs from moving in response to the spring bias;
each limb having a loop arranged so that the loops can engage opposite regions of an inner perimeter of an iris;
while the limbs are in the inter-engaged juxtaposition, they can be inserted through a single incision in the eye; and
as the limbs are inserted into the eye, the limbs are disengaged from each other to respond to the spring bias from the head spring to move the limbs apart and urge the loops against the inner perimeter of the iris to dilate the iris.

10. The dilator of claim 9 wherein the dilator is molded of polymeric spring material shaped to exert different edge spring biases in different regions of the dilator.

11. The dilator of claim 9 wherein the latching of the limbs is arranged to disengage during the insertion of the limbs into the eye.

12. The dilator of claim 9 wherein the loops are inclined relative to a plane of the limbs.
